(19) United States
(12) Patent Application Publication
(10) Pub. No.: US 2006/0205714 A1
(43) Pub. Date: Sep. 14, 2006

(54) Method of synthesis and isolation of solid N-desmethylclozapine and crystalline forms thereof

(76) Inventors: Bo-Ragnar Tolf, Malmo (SE); Mikkel Boas Thygesen, Copenhagen East (DK); Fritz Blatter, Reinach (CH); Jorg Berghausen, Lorrach (DE)

Correspondence Address: KNOBBE MARTENS OLSON & BEAR LLP, 2040 MAIN STREET, FOURTEENTH FLOOR, IRVINE, CA, 92614, US

(21) Appl. No.: 11/416,673

(22) Filed: May 3, 2006

Related U.S. Application Data
(63) Continuation of application No. 11/096,756, filed on Mar. 31, 2005.
(60) Provisional application No. 60/558,881, filed on Apr. 1, 2004.

(51) Int. Cl. A61K 31/551

(52) U.S. Cl. 514/220

(57) ABSTRACT

Disclosed herein are crystalline Forms A, B, C, D, and E of N-desmethylclozapine, methods of preparing the same, pharmaceutical compositions comprising the same, and methods of therapeutic treatment involving N-desmethylclozapine polymorphic forms.

### RELATED APPLICATIONS

The present application is a continuation of U.S. application Ser. No. 11/096,756, by Tolf et al., filed Mar. 31, 2005, and entitled “METHOD OF SYNTHESIS AND ISOLATION OF SOLID N-DESMETHYLCLOZAPINE AND CRYSTALLINE FORMS THEREOF” which in turn claims priority to the U.S. Provisional Application Ser. No. 60/558,881, filed Apr. 1, 2004, by Tolf et al., and entitled “METHOD OF SYNTHESIS AND ISOLATION OF POLYMORPHS OF N-DESMETHYLCLOZAPINE,” both of which are incorporated herein by reference in their entirety.

### FIELD OF THE INVENTION

The present invention relates to various crystalline forms of N-desmethylclozapine, processes for the preparation of the same, and methods of treating disease using the same.

### BACKGROUND TO THE INVENTION

The physiological actions of the hormone/neurotransmitter acetylcholine are mediated, in part, by muscarinic acetylcholine receptors. Muscarinic receptors comprise a family of five (M1-M5) transmembrane proteins that mediate slow, modulatory signalling in cells and tissues expressing these genes. Muscarinic receptors are the targets of a number of therapeutically useful agents. Peripherally, muscarinic receptors mediate the actions of acetylcholine in the parasympathetic nervous system. Peripherally acting muscarinic receptor agonists are therapuetically useful in lowering intra-ocular pressure in patients with glaucoma. Compounds that potentiate the central actions of acetylcholine as well as centrally acting muscarinic receptor agonists have both demonstrated clinical utility in the treatment of a number of neuropsychiatric diseases.

The actions of acetylcholine are terminated by degradation of the molecule by acetylcholinesterase enzymes. Inhibition of these enzymes within the central nervous system leads to increased concentrations of acetylcholine at muscarinic receptors. A number of acetylcholinesterase inhibitors have been developed and are in routine clinical use as cognitive enhancing agents in dementia.

A number of centrally acting muscarinic agonist have been the subject of clinical testing. One of these, Xanomeline, has been shown to possess efficacy in controlling psychosis and related behavioral disturbances observed in Alzheimer's Disease patients. Further, it has recently been demonstrated that xanomeline is efficacious in treating schizophrenia. Interestingly, it displayed efficacy against both positive and negative symptoms, and did not induce adverse motoric effects in initial clinical studies in schizophrenics. These data suggest that compounds with muscarinic receptor agonist properties are likely to be efficacious in treating the behavioral disturbances common to neurodegenerative disease such as Alzheimers Disease and as antipsychotics to treat human psychoses, but only if they are tolerated in these patient populations. Additionally, muscarinic receptor agonists have shown activity in pre-clinical models of neuropathic pain states.

N-desmethylclozapine (NDMC), also known by its chemical name 8-chloro-11-(1-piperazinyl)-5H-dibenzo[b,e]-[1,4]diazepin, has the following formula:

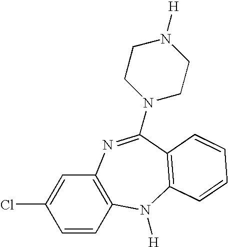

NDMC has been shown to be effective in the treatment of psychosis and other neuropsychiatric disorders. See, International Publication WO 2004/064753 and Dave Weiner et al., Psychopharmacology 2004, 177, 207-216, both of which are incorporated herein by reference in their entirety. Several methods of synthesizing NDMC have also been disclosed. See, for example, International Publication WO 2004/064753, Ger. Patent No. 2316438, and Ben Capuano, Molecules 1999, 4, 329-332, all of which are incorporated herein by reference in their entirety. There is however a need in the art for crystalline NDMC of high purity, and methods of preparing the same, for the preparation of pharmaceutical compositions.

### SUMMARY OF THE INVENTION

Disclosed herein are crystalline Forms A, B, C, D, and E of N-desmethylclozapine, methods of preparing the same, pharmaceutical compositions comprising the same, and methods of therapeutic treatment involving N-desmethylclozapine polymorphic forms.

In one aspect, disclosed herein is a crystalline N-desmethylclozapine. In another aspect, disclosed herein is composition of matter comprising crystalline N-desmethylclozapine.

In another aspect, disclosed herein is a crystalline N-desmethylclozapine substantially free of amorphous N-desmethylclozapine. In one embodiment, the crystalline N-desmethylclozapine comprises less than 30% amorphous N-desmethylclozapine. In another embodiment, the crystalline N-desmethylclozapine comprises less than 25% amorphous N-desmethylclozapine. In another embodiment, the crystalline N-desmethylclozapine comprises less than 20% amorphous N-desmethylclozapine. In another embodiment, the crystalline N-desmethylclozapine comprises less than 15% amorphous N-desmethylclozapine. In another embodiment, the crystalline N-desmethylclozapine comprises less than 10% amorphous N-desmethylclozapine. In another embodiment, the crystalline N-desmethylclozapine comprises less than 5% amorphous N-desmethylclozapine.

In another aspect, disclosed herein is a crystalline N-desmethylclozapine Form A. In one embodiment, the crystalline N-desmethylclozapine produces a powder X-ray diffraction pattern with interplanar d-spacings of 9.9, 6.9, 6.5, 6.3, 6.1, 5.57, 5.09, 4.94, 4.61, 4.47, 4.38, 4.01, 3.74, 3.66, 3.55, 3.45, 3.33, 3.21, 3.08, 3.03, 2.80, and 2.67 (Å). In another embodiment, the crystalline N-desmethylclozapine produces a powder X-ray diffraction pattern with interplanar d-spacings of 6.5, 6.3, 5.57, 5.09, 4.47, 4.38, 4.01, 3.74, 3.66, 3.55, 3.33, 3.21, and 3.08 (Å). In another embodiment, the crystalline N-desmethylclozapine produces a powder X-ray diffraction pattern with interplanar d-spacings of 5.57, 5.09, 4.01, 3.66, 3.55, 3.21, and 3.08 (Å). In another embodiment, the crystalline N-desmethylclozapine produces a powder X-ray diffraction pattern with reflections at 8.9, 12.8, 13.6, 14.0, 14.6, 15.9, 17.4, 17.9, 19.2, 19.9, 20.3, 22.1, 23.8, 24.35, 25.1, 25.8, 26.7, 27.8, 29.0, 29.4, 32.0, and 33.5° 2θ. In another embodiment, the crystalline N-desmethylclozapine produces a powder X-ray diffraction pattern with reflections at 13.6, 14.0, 15.9, 17.4, 19.9, 20.3, 22.1, 23.8, 24.35, 25.1, 26.7, 27.8, and 29.0° 2θ. In another embodiment, the crystalline N-desmethylclozapine produces a powder X-ray diffraction pattern with reflections at 15.9, 17.4, 22.1, 24.35, 25.1, and 27.8° 2θ.

In another aspect, disclosed herein is a crystalline N-desmethylclozapine Form B. In one embodiment, the crystalline N-desmethylclozapine produces a powder X-ray diffraction pattern with interplanar d-spacings of 8.9, 7.7, 7.1, 6.5, 5.94, 5.85, 5.76, 5.30, 5.17, 4.90, 4.67, 4.48, 4.17, 3.93, 3.87, 3.72, 3.68, 3.55, 3.44, 3.36, 3.26, 3.20, 3.06, 2.75, 2.73, 2.49, 2.45, 2.37, and 2.34 (Å). In another embodiment, the crystalline N-desmethylclozapine produces a powder X-ray diffraction pattern with interplanar d-spacings of 8.9, 7.7, 7.1, 6.5, 5.94, 5.85, 5.76, 5.30, 5.17, 4.90, 4.67, 4.17, 3.93, 3.87, 3.72, 3.68, 3.55, 3.44, 3.26, 3.20, 3.06, 2.75, 2.73, 2.49, 2.45, 2.37, and 2.34 (Å) are particularly characteristic. In another embodiment, the crystalline N-desmethylclozapine produces a powder X-ray diffraction pattern with interplanar d-spacings of 7.1, 5.94, 5.30, 5.17, 4.17, 3.93, 3.72, 3.68, 3.44, 3.26, and 3.06 (Å). In another embodiment, the crystalline N-desmethylclozapine produces a powder X-ray diffraction pattern with reflections at 9.9, 11.4, 12.5, 13.7, 14.9, 15.1, 15.4, 16.7, 17.2, 18.1, 1.9.0, 19.8, 21.3, 22.6, 23.0, 23.9, 24.2, 25.0, 25.9, 26.5, 27.3, 27.9, 29.1, 32.5, 32.8, 36.0, 36.7, 38.0, and 38.5° 2θ. In another embodiment, the crystalline N-desmethylclozapine produces a powder X-ray diffraction pattern with reflections at 9.9, 11.4, 12.5, 13.7, 14.9, 15.1, 15.4, 16.7, 17.2, 18.1, 19.0, 21.3, 22.6, 23.0, 23.9, 24.2, 25.0, 25.9, 27.3, 27.9, 29.1, 32.5, 32.8, 36.0, 36.7, 38.0, and 38.5° 2θ. In another embodiment, the crystalline N-desmethylclozapine produces a powder X-ray diffraction pattern with reflections at 12.5, 14.9, 16.7, 17.2, 21.3, 22.6, 23.9, 24.2, 25.9, 27.3, 29.1° 2θ.

In another aspect, disclosed herein is a crystalline N-desmethylclozapine Form C. In one embodiment, the crystalline N-desmethylclozapine produces a powder X-ray diffraction pattern with interplanar d-spacings of 14.2, 13.7, 12.2, 11.7, 7.9, 6.9, 6.4, 5.83, 5.42, 5.17, 4.95, 4.59, 4.46, 3.94, and 3.63 (Å). In another embodiment, the crystalline N-desmethylclozapine produces a powder X-ray diffraction pattern with interplanar d-spacings of 12.2, 5.17, 4.95, 4.59, 4.46, 3.94, and 3.63 (Å). In another embodiment, the crystalline N-desmethylclozapine produces a powder X-ray diffraction pattern with interplanar d-spacings of 4.95, 4.59, 4.46, and 3.94 (Å). In another embodiment, the crystalline N-desmethylclozapine produces a powder X-ray diffraction pattern with reflections at 6.2, 6.5, 7.2, 7.6, 11.3, 12.8, 13.9, 15.2, 16.3, 17.1, 17.9, 19.3, 19.9, 22.5, and 24.5° 2θ. In another embodiment, the crystalline N-desmethylclozapine produces a powder X-ray diffraction pattern with reflections at 7.2, 17.1, 17.9, 19.3, 19.9, 22.5, and 24.5° 2θ. In another embodiment, the crystalline N-desmethylclozapine produces a powder X-ray diffraction pattern with reflections at 17.9, 19.3, 19.9, and 22.5° 2θ.

In another aspect, disclosed herein is a crystalline N-desmethylclozapine Form D. In one embodiment, the crystalline N-desmethylclozapine produces a powder X-ray diffraction pattern with interplanar d-spacings of 8.6, 7.6, 7.0, 6.4, 6.1, 5.81, 5.52, 5.24, 5.03, 4.95, 4.73, 4.20, 4.04, 3.90, 3.80, 3.70, 3.63, 3.50, 3.42, 3.37, 3.33, 3.26, 3.20, 3.13, 3.04, and 2.71 (Å). In another embodiment, the crystalline N-desmethylclozapine produces a powder X-ray diffraction pattern with interplanar d-spacings of 8.6, 7.0, 6.4, 5.81, 5.52, 5.24, 5.03, 4.95, 4.73, 4.20, 4.04, 3.90, 3.80, 3.70, 3.63, 3.50, 3.42, 3.37, 3.33, 3.26, 3.20, 3.13, 3.04, and 2.71 (Å). In another embodiment, the crystalline N-desmethylclozapine produces a powder X-ray diffraction pattern with interplanar d-spacings of 7.0, 5.24, 5.03, 4.20, 4.04, 3.80, 3.70, 3.63, 3.37, and 3.04 (Å) are most characteristic. In another embodiment, the crystalline N-desmethylclozapine produces a powder X-ray diffraction pattern with reflections at 10.3, 11.6, 12.6, 13.8, 14.5, 15.2, 16.0, 16.9, 17.6, 17.9, 18.7, 21.1, 22.0, 22.8, 23.4, 24.0, 24.5, 25.4, 26.1, 26.4, 26.8, 27.3, 27.8, 28.5, 29.3, and 33.0° 2θ. In another embodiment, the crystalline N-desmethylclozapine produces a powder X-ray diffraction pattern with reflections at 10.3, 12.6, 13.8, 15.2, 16.0, 16.9, 17.6, 17.9, 18.7, 21.1, 22.0, 22.8, 23.4, 24.0, 24.5, 25.4, 26.1, 26.4, 26.8, 27.3, 27.8, 28.5, 29.3, and 33.0° 2θ. In another embodiment, the crystalline N-desmethylclozapine produces a powder X-ray diffraction pattern with reflections at 12.6, 16.9, 17.6, 21.1, 22.0, 23.4, 24.0, 24.5, 26.4, and 29.3° 2θ.

In another aspect, disclosed herein is a crystalline N-desmethylclozapine Form E. In one embodiment, the crystalline N-desmethylclozapine produces a powder X-ray diffraction pattern with interplanar d-spacings of 12.6, 11.8, 11.0, 7.3, 7.0, 6.7, 6.4, 5.90, 5.60, 5.35, 4.95, 4.62, 4.44, 4.01, 3.94, 3.75, 3.37, and 3.00 (Å). In another embodiment, the crystalline N-desmethylclozapine produces a powder X-ray diffraction pattern with interplanar d-spacings of 4.95, 4.62, 4.44, 4.01, 3.94, and 3.75 (Å). In another embodiment, the crystalline N-desmethylclozapine produces a powder X-ray diffraction pattern with interplanar d-spacings of 4.95, 4.62, and 4.44 (Å) are most characteristic. In another embodiment, the crystalline N-desmethylclozapine produces a powder X-ray diffraction pattern with reflections at 7.0, 7.5, 8.0, 12.1, 12.7, 13.3, 13.9, 15.0, 15.8, 16.6, 17.9, 19.2, 20.0, 22.1, 22.6, 23.7, 26.4, and 29.7° 2θ. In another embodiment, the crystalline N-desmethylclozapine produces a powder X-ray diffraction pattern with reflections at 17.9, 19.2, 20.0, 22.1, 22.6, and 23.7° 2θ. In another embodiment, the crystalline N-desmethylclozapine produces a powder X-ray diffraction pattern with reflections at 17.9, 19.2, and 20.0° 2θ.

In another aspect, disclosed herein is a pharmaceutical composition comprising crystalline N-desmethylclozapine and a pharmaceutically acceptable carrier, eluent, or excipient. In one embodiment, the crystalline N-desmethylclozapine is substantially free of amorphous N-desmethylclozapine. In another embodiment, the crystalline N-desmethylclozapine is N-desmethylclozapine Form A. In another embodiment, the crystalline N-desmethylclozapine is N-desmethylclozapine Form B. In another embodiment, the crystalline N-desmethylclozapine is N-desmethylclozapine Form C. In another embodiment, the crystalline N-desmethylclozapine is N-desmethylclozapine Form D. In another embodiment, the crystalline N-desmethylclozapine is N-desmethylclozapine Form E.

### BRIEF DESCRIPTION OF THE DRAWINGS

FIG. 1 is a characteristic X-ray powder diffraction pattern of N-desmethylclozapine Form A.

FIG. 2 is a characteristic X-ray powder diffraction pattern of N-desmethylclozapine Form B (monohydrate).

FIG. 3 is a characteristic X-ray powder diffraction pattern of N-desmethylclozapine Form C.

FIG. 4 is a characteristic X-ray powder diffraction pattern of N-desmethylclozapine Form D.

FIG. 5 is a characteristic X-ray powder diffraction pattern of N-desmethylclozapine Form E.

### DETAILED DESCRIPTION OF THE INVENTION

Synthesis of N-Desmethylclozapine

In the first aspect, disclosed herein is a process for the preparation of 8-chloro-11-(1-piperazinyl)-5H-dibenzo[b,e][1,4]diazepin (N-desmethylclozapine, NDMC) of formula I 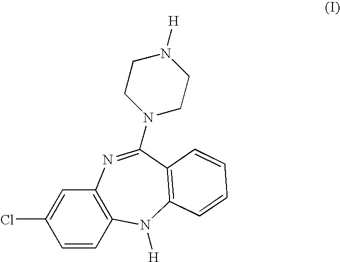
comprising reacting a compound of formula II 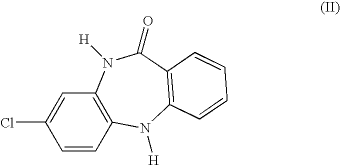
with piperazine in the presence of a metal salt as Lewis acid and an inert solvent, wherein, preferably, the solvent comprises an aromatic ring.

In some embodiments, there is a one-to-one molar ratio between the amount of piperazine and the amount of the compound of formula II. In other embodiments, piperazine is used in excess. In certain of these embodiments, piperizine is added in at least 6 equivalents, or at least 8 equivalents, or at least 10 equivalents of the amount of the compound of formula II.

In certain embodiments, the aromatic ring of the solvent is unsubstituted. In other embodiments, the aromatic ring is substituted with at least one substituent selected from the group consisting of chlorine, fluorine, C₁-C₁₀ alkyl, C₁-C₁₀ alkoxy, and aryloxy. In some embodiments, the solvent is selected form the group consisting of benzene, fluorobenzene, difluorobenzene, chlorobenzene, dichlorobenzene, toluene, xylene, methoxybenzene, and dimethoxybenzene. In another embodiment, the solvent is anisole.

A wide variety of metal salts as Lewis acids are suitable for use in the processes disclosed herein. In some embodiments, the metal cation of the metal salt is selected from the group consisting of B, Al, Sn, Pb, Sb, Bi, Ti, Zr and Hf. In some embodiments, the metal is Ti. In further embodiments, the Ti is in its fourth oxidation state, i.e, it is present as Ti(IV). In some embodiments, the anion of the metal salt is the conjugate base of an inorganic or organic acid selected from the group consisting of HCl, HBr, HI, H₂SO₄, HNO₃, H₃PO₄, formic acid, acetic acid, oxalic acid, trifluoromethanesulfonic acid, trifluoromethanesulfonic acid, benzene sulfonic acid, toluolsulfonic acid, benzene phosponics acid. Especially preferred are halogenides such as chlorides and bromides. In some embodiments, the metal salt is TiCl₄.

The Lewis acid may be present in equivalent amounts to the compound of Formula II. In some embodiments, the Lewis acid is present in excess. In certain of these embodiments, the Lewis acid is present in at least 1.5 equivalents, or at least 2 equivalents, or at least 3 equivalents of the compound of formula II.

The reaction temperature may be in the range of 50 to 200° C. and preferably 80 to 150° C.

The processes disclosed herein can be carried out in feeding a suitable reactor at about room temperature with the solvent followed by the addition of the Lewis acid and then piperazine. The resulting suspension is then warmed to a temperature in the range of 40 to 70° C. The compound of formula II is then added at this temperature. In some embodiments, the compound of formula II is added in portions and under external cooling to avoid a higher inner temperature due to an exothermic reaction. In other embodiments, the entire amount of the compound of formula II to be reacted is added at once.

In yet other embodiments, the compound of formula II is added to the reaction mixture prior to piperazine. In yet other embodiments, the Lewis acid is added prior to piperazine. In further embodiments, the Lewis acid is the last ingredient added to the reaction mixture.

After completion of the addition, the reaction mixture can be heated to a temperature of up to 200° C. and stirred at this temperature for a period of time until the reaction is completed. The reaction time may last up to 6 hours. However, in some embodiments, the reaction time lasts up to 2 hours. In further embodiments, the reaction time lasts longer than 6 hours. In some embodiments, the reaction stopped before it reaches completion. The extent of the conversion of the compound of formula II may be determined by HPLC, or any other characterization tool, such as TLC, UV-Vis, NMR, or IR, to define the termination of the reaction at preferably about 99% conversion or more.

Following the above steps, the reaction mixture is cooled to a temperature of about −10 to 5° C. and base, such as an alkaline or alkaline earth metal oxides or hydroxides, such as LiOH, NaOH, KOH, CaO, MgO, Mg(OH)₂, Ca(OH)₂, or an alkaline earth metal carbonate, such as Na₂CO₃, NaHCO₃, K₂CO₃, KHCO₃, is added to the reaction mixture. The amount of the base is in excess, for example up to 6 equivalents, of the Lewis acid. The Lewis acid is thus converted to a filterable salt, which is then filtered away. The desired N-desmethylclozapine is then extracted and purified by crystallization and further dried. N-desmethylclozapine is obtained as a yellow solid, which shows different melting ranges depending essentially on the drying conditions and water content of the product.

Crystalline N-Desmethylclozapine

In another aspect, disclosed herein is crystalline N-desmethylclozapine. In another aspect, disclosed herein is a composition of matter comprising crystalline N-desmethylclozapine. In yet another aspect, disclosed herein is crystalline N-desmethylclozapine substantially free of amorphous N-desmethylclozapine.

In some embodiments, by “substantially free of amorphous N-desmethylclozapine” it is meant that the N-desmethylclozapine sample comprises less than 30% amorphous N-desmethylclozapine. In other embodiments, by “substantially free of amorphous N-desmethylclozapine” it is meant that the N-desmethylclozapine sample comprises less than 25% amorphous N-desmethylclozapine. In other embodiments, by “substantially free of amorphous N-desmethylclozapine” it is meant that the N-desmethylclozapine sample comprises less than 20% amorphous N-desmethylclozapine. In other embodiments, by “substantially free of amorphous N-desmethylclozapine” it is meant that the N-desmethylclozapine sample comprises less than 15% amorphous N-desmethylclozapine. In other embodiments, by “substantially free of amorphous N-desmethylclozapine” it is meant that the N-desmethylclozapine sample comprises less than 10% amorphous N-desmethylclozapine. In other embodiments, by “substantially free of amorphous N-desmethylclozapine” it is meant that the N-desmethylclozapine sample comprises less than 5% amorphous N-desmethylclozapine.

In certain embodiments, the crystalline N-desmethylclozapine has a melting range of 176.4-177.6° C. In some embodiments, the melting range is determined with a B-545 melting point apparatus.

Polymorphs of Crystalline N-Desmethylclozapine

In another aspect, disclosed herein are various polymorphs of crystalline N-desmethylclozapine. As compared with the known amorphous form of N-desmethylclozapine, these polymorphs are surprisingly easier to handle and exhibit greater purity and longer shelf-life. Because of their purity and ease of handling, these polymorphs are better suited to be used in pharmaceutical compositions.

Form A

In one aspect, disclosed herein is N-desmethylclozapine Form A. N-desmethylclozapine Form A produces a powder X-ray diffraction pattern with interplanar d-spacings of 9.9, 6.9, 6.5, 6.3, 6.1, 5.57, 5.09, 4.94, 4.61, 4.47, 4.38, 4.01, 3.74, 3.66, 3.55, 3.45, 3.33, 3.21, 3.08, 3.03, 2.80, and 2.67 (Å). Of these the d-spacings of 6.5, 6.3, 5.57, 5.09, 4.47, 4.38, 4.01, 3.74, 3.66, 3.55, 3.33, 3.21, and 3.08 (Å) are particularly characteristic. Of these the d-spacings of 5.57, 5.09, 4.01, 3.66, 3.55, 3.21, and 3.08 (Å) are most characteristic.

N-desmethylclozapine Form A is also characterized by a powder X-ray diffraction pattern with reflections at 8.9, 12.8, 13.6, 14.0, 14.6, 15.9, 17.4, 17.9, 19.2, 19.9, 20.3, 22.1, 23.8, 24.35, 25.1, 25.8, 26.7, 27.8, 29.0, 29.4, 32.0, and 33.5° 2θ. Of these reflections at 13.6, 14.0, 15.9, 17.4, 19.9, 20.3, 22.1, 23.8, 24.35, 25.1, 26.7, 27.8, and 29.0° 2θ are particularly characteristic. Of these, reflections at 15.9, 17.4, 22.1, 24.35, 25.1, and 27.8° 2θ are most characteristic.

In some embodiments, N-desmethylclozapine Form A exhibits a melting point of 177° C., determined with Differential Scanning Calorimetry (DSC) at a heating rate of 10° C./minute. The enthalpy of fusion is about 96 J/g.

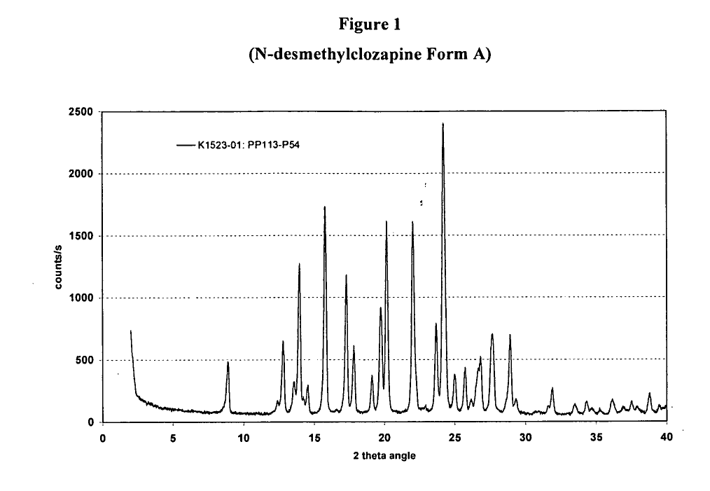

The data from powder X-ray diffraction analysis for N-desmethylclozapine Form A is given in Table 1, below, and in FIG. 1.

TABLE 1
D-Spacings for N-desmethylclozapine Form A

| Angle [°2θ] | d-spacings [Å] | Intensity (qualitative) |
|---|---|---|
| 8.9 | 9.9 | w |
| 12.8 | 6.9 | w |
| 13.6 | 6.5 | m |
| 14.0 | 6.3 | m |
| 14.6 | 6.1 | w |
| 15.9 | 5.57 | s |
| 17.4 | 5.09 | s |
| 17.9 | 4.94 | w |
| 19.2 | 4.61 | w |
| 19.9 | 4.47 | m |
| 20.3 | 4.38 | m |
| 22.1 | 4.01 | s |
| 23.8 | 3.74 | m |
| 24.35 | 3.66 | vs |
| 25.1 | 3.55 | s |
| 25.8 | 3.45 | w |
| 26.7 | 3.33 | m |
| 27.8 | 3.21 | s |
| 29.0 | 3.08 | m |
| 29.4 | 3.03 | w |
| 32.0 | 2.80 | w |
| 33.5 | 2.67 | w |

Here and in the following the abbreviations in brackets mean: (vs)=very strong intensity; (s)=strong intensity; (m)=medium intensity; (w)=weak intensity and (vw)=very weak intensity.

N-desmethylclozapine Form A forms at ambient temperatures and exhibits excellent physical and chemical stability properties. Form A is even very stable under humid atmosphere. It does not convert to the hydrated forms or other crystalline forms even when stored at high, such as at 75% or 90%, relative humidity in air at elevated temperature. Form A shows better water solubility than crystal form B. Form A can be prepared as a solid powder with desired medium particle size range which is typically ranging from 1 μm to about 500 μm. Form A is especially suitable for the formulation of solid drugs, because handling does not require use of inert atmosphere.

Form B

In another aspect, disclosed herein is N-desmethylclozapine Form B. N-desmethylclozapine Form B is a hydrated form having a water content of about 5.4%, which corresponds to the monohydrate.

N-desmethylclozapine Form B produces a powder X-ray diffraction pattern with interplanar d-spacings of 8.9, 7.7, 7.1, 6.5, 5.94, 5.85, 5.76, 5.30, 5.17, 4.90, 4.67, 4.48, 4.17, 3.93, 3.87, 3.72, 3.68, 3.55, 3.44, 3.36, 3.26, 3.20, 3.06, 2.75, 2.73, 2.49, 2.45, 2.37, and 2.34 (Å). Of these the d-spacings of 8.9, 7.7, 7.1, 6.5, 5.94, 5.85, 5.76, 5.30, 5.17, 4.90, 4.67, 4.17, 3.93, 3.87, 3.72, 3.68, 3.55, 3.44, 3.26, 3.20, 3.06, 2.75, 2.73, 2.49, 2.45, 2.37, and 2.34 (Å) are particularly characteristic. Of these the d-spacings of 7.1, 5.94, 5.30, 5.17, 4.17, 3.93, 3.72, 3.68, 3.44, 3.26, and 3.06 (Å) are most characteristic.

N-desmethylclozapine Form B is also characterized by a powder X-ray diffraction pattern with reflections at 9.9, 11.4, 12.5, 13.7, 14.9, 15.1, 15.4, 16.7, 17.2, 18.1, 19.0, 19.8, 21.3, 22.6, 23.0, 23.9, 24.2, 25.0, 25.9, 26.5, 27.3, 27.9, 29.1, 32.5, 32.8, 36.0, 36.7, 38.0, and 38.5° 2θ. Of these reflections at 9.9, 11.4, 12.5, 13.7, 14.9, 15.1, 15.4, 16.7, 17.2, 18.1, 19.0, 21.3, 22.6, 23.0, 23.9, 24.2, 25.0, 25.9, 27.3, 27.9, 29.1, 32.5, 32.8, 36.0, 36.7, 38.0, and 38.5° 2θ are particularly characteristic. Of these, reflections at 12.5, 14.9, 16.7, 17.2, 21.3, 22.6, 23.9, 24.2, 25.9, 27.3, 29.1° 2θ are most characteristic.

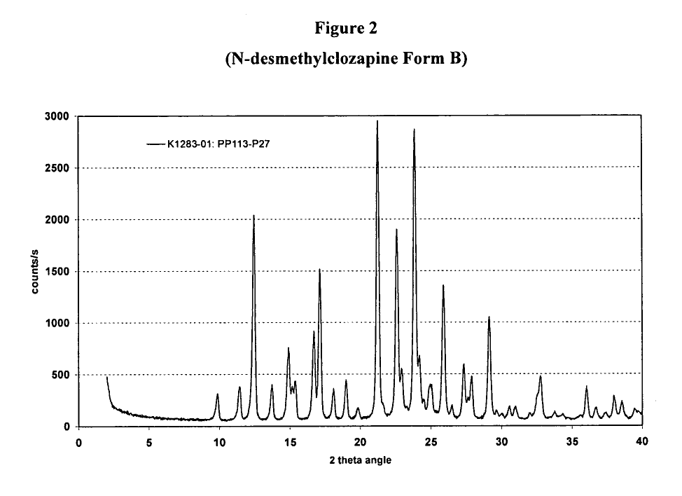

The data from powder X-ray diffraction analysis for N-desmethylclozapine Form B is given in Table 2, below, and in FIG. 2.

TABLE 2
D-Spacings for form B

| Angle [°2θ] | d-spacings [Å] | Intensity (qualitative) |
|---|---|---|
| 9.9 | 8.9 | m |
| 11.4 | 7.7 | m |
| 12.5 | 7.1 | vs |
| 13.7 | 6.5 | m |
| 14.9 | 5.94 | s |
| 15.1 | 5.85 | m |
| 15.4 | 5.76 | m |
| 16.7 | 5.30 | s |
| 17.2 | 5.17 | vs |
| 18.1 | 4.90 | m |
| 19.0 | 4.67 | m |
| 19.8 | 4.48 | w |
| 21.3 | 4.17 | vs |
| 22.6 | 3.93 | vs |
| 23.0 | 3.87 | m |
| 23.9 | 3.72 | vs |
| 24.2 | 3.68 | s |
| 25.0 | 3.55 | m |
| 25.9 | 3.44 | s |
| 26.5 | 3.36 | w |
| 27.3 | 3.26 | s |
| 27.9 | 3.20 | m |
| 29.1 | 3.06 | s |
| 32.5 | 2.75 | m |
| 32.8 | 2.73 | m |
| 36.0 | 2.49 | m |
| 36.7 | 2.45 | m |
| 38.0 | 2.37 | m |
| 38.5 | 2.34 | m |

Form B is a very stable hydrate even when stored at high, such as 75% or 90%, relative humidity in air and at elevated temperature. No conversion to other crystalline forms or hydrates was observed. The melting point of Form B is 149° C., determined with Differential Scanning Calorimetry (DSC) at a heating rate of 10° C./minute. Form B is specially water soluble. Form B can be prepared as a solid powder with desired medium particle size range which is typically ranging from 1 μm to about 500 μm. Form B is especially suitable for the formulation of solid drugs, because handling does not require use of inert atmosphere.

It was also found that crystal forms A and B can be formed as mixtures when prepared according to the process of this invention or when crystallized under humid conditions. These mixtures are also very stable and therefore especially suitable for the formulation of solid drugs. Another object of the invention is a composition comprising a mixture of crystalline form A and crystalline form B of N-desmethylclozapine monohydrate. The ratio of the two forms is not critical.

Form C

In another aspect, disclosed herein is N-desmethylclozapine Form C. N-desmethylclozapine Form C can be obtained, when a solution of N-desmethylclozapine in polar solvents or solvent mixtures is completely evaporated.

N-desmethylclozapine Form C produces a powder X-ray diffraction pattern with interplanar d-spacings of 14.2, 13.7, 12.2, 11.7, 7.9, 6.9, 6.4, 5.83, 5.42, 5.17, 4.95, 4.59, 4.46, 3.94, and 3.63 (Å). Of these the d-spacings of 12.2, 5.17, 4.95, 4.59, 4.46, 3.94, and 3.63 (Å) are particularly characteristic. Of these the d-spacings of 4.95, 4.59, 4.46, and 3.94 (Å) are most characteristic.

N-desmethylclozapine Form C is also characterized by a powder X-ray diffraction pattern with reflections at 6.2, 6.5, 7.2, 7.6, 11.3, 12.8, 13.9, 15.2, 16.3, 17.1, 17.9, 19.3, 19.9, 22.5, and 24.5° 2θ. Of these reflections at 7.2, 17.1, 17.9, 19.3, 19.9, 22.5, and 24.5° 2θ are particularly characteristic. Of these, reflections at 17.9, 19.3, 19.9, and 22.5° 2θ are most characteristic.

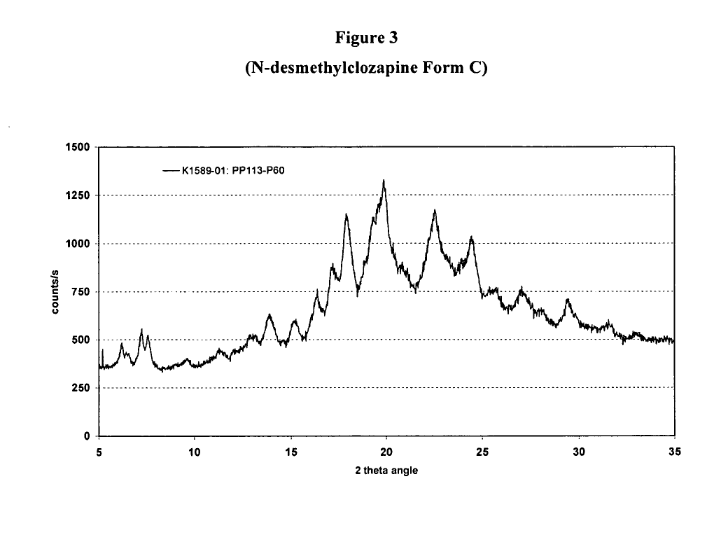

The data from powder X-ray diffraction analysis for N-desmethylclozapine Form C is given in Table 3, below, and in FIG. 3.

TABLE 3
D-Spacings for form C

| Angle [°2θ] | d-spacings [Å] | Intensity (qualitative) |
|---|---|---|
| 6.2 | 14.2 | w |
| 6.5 | 13.7 | w |
| 7.2 | 12.2 | m |
| 7.6 | 11.7 | w |
| 11.3 | 7.9 | w |
| 12.8 | 6.9 | w |
| 13.9 | 6.4 | w |
| 15.2 | 5.83 | w |
| 16.3 | 5.42 | w |
| 17.1 | 5.17 | m |
| 17.9 | 4.95 | s |
| 19.3 | 4.59 | s |
| 19.9 | 4.46 | vs |
| 22.5 | 3.94 | s |
| 24.5 | 3.63 | m |

Form D

In another aspect, disclosed herein is N-desmethylclozapine Form D. N-desmethylclozapine Form B can be transformed in a Form D under controlled dehydration conditions.

N-desmethylclozapine Form D produces a powder X-ray diffraction pattern with interplanar d-spacings of 8.6, 7.6, 7.0, 6.4, 6.1, 5.81, 5.52, 5.24, 5.03, 4.95, 4.73, 4.20, 4.04, 3.90, 3.80, 3.70, 3.63, 3.50, 3.42, 3.37, 3.33, 3.26, 3.20, 3.13, 3.04, and 2.71 (Å). Of these the d-spacings of 8.6, 7.0, 6.4, 5.81, 5.52, 5.24, 5.03, 4.95, 4.73, 4.20, 4.04, 3.90, 3.80, 3.70, 3.63, 3.50, 3.42, 3.37, 3.33, 3.26, 3.20, 3.13, 3.04, and 2.71 (Å) are particularly characteristic. Of these the d-spacings of 7.0, 5.24, 5.03, 4.20, 4.04, 3.80, 3.70, 3.63, 3.37, and 3.04 (Å) are most characteristic.

N-desmethylclozapine Form D is also characterized by a powder X-ray diffraction pattern with reflections at 10.3, 11.6, 12.6, 13.8, 14.5, 15.2, 16.0, 16.9, 17.6, 17.9, 18.7, 21.1, 22.0, 22.8, 23.4, 24.0, 24.5, 25.4, 26.1, 26.4, 26.8, 27.3, 27.8, 28.5, 29.3, and 33.0° 2θ. Of these reflections at 10.3, 12.6, 13.8, 15.2, 16.0, 16.9, 17.6, 17.9, 18.7, 21.1, 22.0, 22.8, 23.4, 24.0, 24.5, 25.4, 26.1, 26.4, 26.8, 27.3, 27.8, 28.5, 29.3, and 33.0° 2θ are particularly characteristic. Of these, reflections at 12.6, 16.9, 17.6, 21.1, 22.0, 23.4, 24.0, 24.5, 26.4, and 29.3° 2θ are most characteristic.

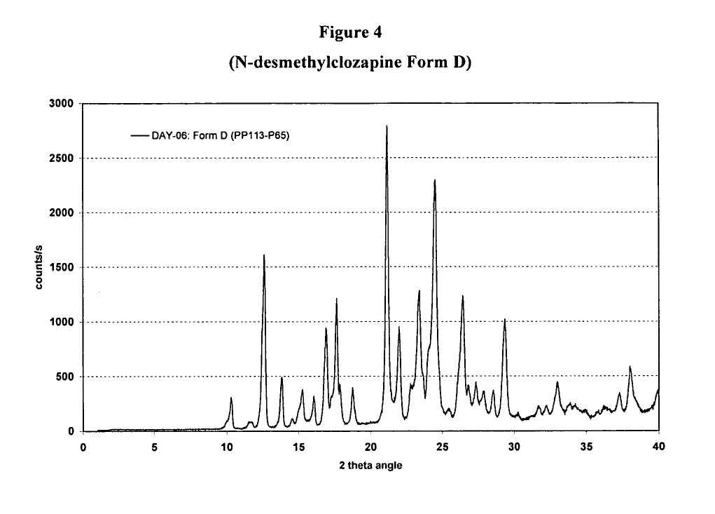

The data from powder X-ray diffraction analysis for N-desmethylclozapine Form D is given in Table 4, below, and in FIG. 4.

TABLE 4
D-Spacings for form D

| Angle [°2θ] | d-spacings [Å] | Intensity (qualitative) |
|---|---|---|
| 10.3 | 8.6 | m |
| 11.6 | 7.6 | w |
| 12.6 | 7.0 | vs |
| 13.8 | 6.4 | m |
| 14.5 | 6.1 | w |
| 15.2 | 5.81 | m |
| 16.0 | 5.52 | m |
| 16.9 | 5.24 | s |
| 17.6 | 5.03 | s |
| 17.9 | 4.95 | m |
| 18.7 | 4.73 | m |
| 21.1 | 4.20 | vs |
| 22.0 | 4.04 | s |
| 22.8 | 3.90 | m |
| 23.4 | 3.80 | s |
| 24.0 | 3.70 | s |
| 24.5 | 3.63 | vs |
| 25.4 | 3.50 | m |
| 26.1 | 3.42 | m |
| 26.4 | 3.37 | s |
| 26.8 | 3.33 | m |
| 27.3 | 3.26 | m |
| 27.8 | 3.20 | m |
| 28.5 | 3.13 | m |
| 29.3 | 3.04 | s |
| 33.0 | 2.71 | m |

N-desmethylclozapine Form D is stable under exclusion of humidity and at ambient temperature. N-desmethylclozapine Form B is formed within hours when N-desmethylclozapine Form D is contacted with humidity. N-desmethylclozapine Form D shows a satisfying solubility in solvents and can be used as starting material for the preparation of other crystal forms.

Form E

In another aspect, disclosed herein is N-desmethylclozapine Form E. N-desmethylclozapine Form E can be obtained, when the solvent tetrahydrofuran is completely evaporated at room temperature.

N-desmethylclozapine Form E produces a powder X-ray diffraction pattern with interplanar d-spacings of 12.6, 11.8, 11.0, 7.3, 7.0, 6.7, 6.4, 5.90, 5.60, 5.35, 4.95, 4.62, 4.44, 4.01, 3.94, 3.75, 3.37, and 3.00 (Å). Of these the d-spacings of 4.95, 4.62, 4.44, 4.01, 3.94, and 3.75 (Å) are particularly characteristic. Of these the d-spacings of 4.95, 4.62, and 4.44 (Å) are most characteristic.

N-desmethylclozapine Form E is also characterized by a powder X-ray diffraction pattern with reflections at 7.0, 7.5, 8.0, 12.1, 12.7, 13.3, 13.9, 15.0, 15.8, 16.6, 17.9, 19.2, 20.0, 22.1, 22.6, 23.7, 26.4, and 29.7° 2θ. Of these reflections at and 17.9, 19.2, 20.0, 22.1, 22.6, and 23.7° 2θ are particularly characteristic. Of these, reflections at 17.9, 19.2, and 20.0° 2θ are most characteristic.

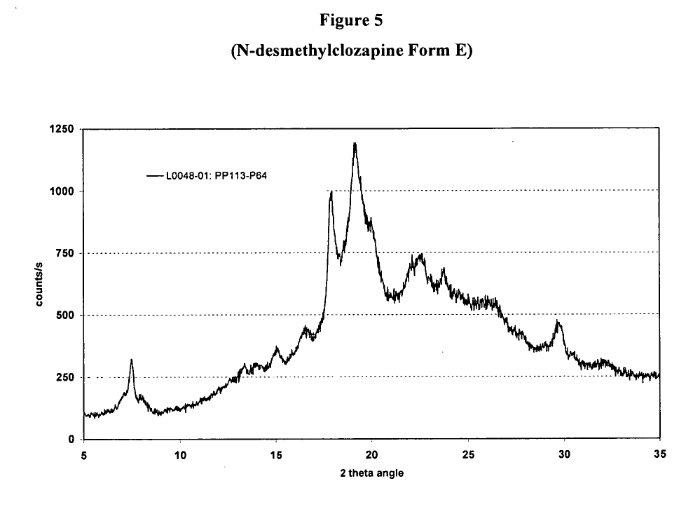

The data from powder X-ray diffraction analysis for N-desmethylclozapine Form E is given in Table 5, below, and in FIG. 5.

TABLE 5
D-Spacings for form E

| Angle [°2θ] | d-spacings [Å] | Intensity (qualitative) |
|---|---|---|
| 7.0 | 12.6 | vw |
| 7.5 | 11.8 | w |
| 8.0 | 11.0 | vw |
| 12.1 | 7.3 | vw |
| 12.7 | 7.0 | vw |
| 13.3 | 6.7 | vw |
| 13.9 | 6.4 | vw |
| 15.0 | 5.90 | vw |
| 15.8 | 5.60 | vw |
| 16.6 | 5.35 | w |
| 17.9 | 4.95 | s |
| 19.2 | 4.62 | vs |
| 20.0 | 4.44 | s |
| 22.1 | 4.01 | m |
| 22.6 | 3.94 | m |
| 23.7 | 3.75 | m |
| 26.4 | 3.37 | w |
| 29.7 | 3.00 | w |

For the preparation of the polymorph forms disclosed here, crystallization techniques well known in the art, such as stirring of a suspension (phase equilibration), precipitation, re-crystallisation, evaporation, solvent like water sorption methods or decomposition of solvates, can be used. Diluted, saturated or super-saturated solutions can be used for crystallization, with or without seeding with suitable nucleating agents. Temperatures up to 100° C. may be applied to form solutions. Cooling to initiate crystallization and precipitation down to −100° C. and preferably down to −30° C. may be applied. Amorphous or crystalline starting materials can be used to prepare solutions or suspensions for the preparation of more stable forms and to achieve higher concentrations in the solutions. The processes may be carried out with or without seeding.

Preparation of Crystal Form A

In one embodiment N-desmethylclozapine Form A is prepared by dissolving crystalline or amorphous N-desmethylclozapine in a suitable solvent or solvent mixture and crystallizing the product by cooling, partial solvent evaporation or addition of non-solvents. The procedure is preferably carried out under conditions that exclude humidity to avoid contaminations with a hydrate. Mixtures of Form A and monohydrate Form B can be formed in the presence of humidity or water in a solvent. Form A can also be prepared by phase equilibration in suitable solvents and ambient temperatures. Examples of suitable solvents include, but are not limited to, ethylacetate, acetonitrile, heptane, ethanol or mixtures thereof. Examples of suitable non-solvents include, but are not limited to, aliphatic hydrocarbons such as hexane, heptane, cyclohexane, methylcyclohexane and aliphatic ethers such as t-butyl methyl ether. Solvent evaporation may be achieved under vacuum or in a dry inert gas stream such as an air stream or a nitrogen stream. Dissolution may be carried out by heating a suspension to temperatures of up to 120° C. or preferably up to 80° C., until a clear solution is obtained.

Disclosed herein is a process for the preparation of N-desmethylclozapine Form A by dissolving any solid-state form including the amorphous form of N-desmethylclozapine in a suitable solvent, which is substantially free of water, optionally evaporating part of said solvent and/or adding a non-polar anti-solvent to precipitate N-desmethylclozapine Form A, or cooling the solution to crystallize and precipitate N-desmethylclozapine Form A.

The solvent is preferably an aliphatic alcohol such as a C₁-C₅ alcohol, an ester of an aliphatic carboxylic acid and alcohol such as C₂-C₄ alkyl esters of acetic acid, or an aliphatic C₂-C₆ ketone such as acetone, methyl propyl ketone, diethyl ketone or methyl i- or t-butyl ketone. The non-polar anti-solvent is preferably an aliphatic hydrocarbon such as petroleum ether, pentane, hexane, heptane, octane, cyclopentane, cyclohexane or methylcyclohexane, or an aliphatic ether such as diethyl ether, methyl propyl ether or dibutyl ether.

In one embodiment, disclosed herein is a process for the preparation of N-desmethylclozapine Form A comprising
a) dissolving solid N-desmethylclozapine in a solvent selected from the group consisting of ethyl acetate, acetonitrile, ethanol, propanol, butanol and heptane, or in mixtures of at least two of said solvents,
b) crystallizing N-desmethylclozapine either by cooling, partial evaporation of solvent, or addition of a non-solvent, wherein said non-solvent is selected from the group consisting of methyl cyclohexane, heptane and methyl t-butyl ether, or a combination of cooling, partial evaporation of solvent, and addition of a non-solvent, and
c) filtering off N-desmethylclozapine Form A and removing residual solvent.

The concentration of N-desmethylclozapine in the solution may be from 5 to 50 and preferably 10 to 40 percent by weight of the solution. Dissolution may be carried out by heating a suspension up to 60° C. until a clear solution is formed.

By “cooling” it is meant lowering the temperature of the mixture to about −20 to 10° C. and more preferably −10 to 5° C. By “partial evaporation” it is meant removing about at least 10 and up to 70 weight percent, preferably at least 20 and up to 60 weight percent, and more preferably at least 30 and up to 50 weight percent of the solvent or solvent mixture. The amount of added non-solvent may be in the range of 5 and up to 60 weight percent and more preferably 10 to 40 weight percent, of the used solved. Residual solvent may be removed under vacuum, in an inert gas flow or both.

Form A may also be prepared by phase equilibration in stirring a suspension of solid N-desmethylclozapine in solvents or solvent mixtures such as heptane/ethyl acetate or t-butyl methyl ether at a temperature of about 10 to 30° C. and preferably 15 to 25° C. for a time sufficient to form N-desmethylclozapine Form A. The treatment may be applied for up to 100, preferably up to 50 and more preferably up to 30 hours.

Preparation of Crystal Form B

In another aspect, disclosed herein is a process for the preparation of N-desmethylclozapine Form B comprising
a) dissolving solid N-desmethylclozapine in a solvent and precipitating the solids at ambient temperature by the addition of water; or
b) stirring a suspension of solid N-desmethylclozapine in water or in a mixture of water and a solvent, and
c) removing water or the mixture of water and a solvent to dryness, or filtering off N-desmethylclozapine Form B of and removing residual water or the mixture of solvent and water.

The concentration of N-desmethylclozapine in the solution may be from 5 to 50 and preferably 10 to 40 percent by weight of the solution. Dissolution may be carried out by heating a suspension up to 60° C. until formation of a clear solution. Prior to the addition of water, the mixture in step a) is then cooled to ambient temperatures, which is preferably about 20 to 25° C. The mixture is then further cooled to about 2 to 15° C. and preferably 5 to 10° C. after the addition of water for a period of time, for example for up to 50 and preferably up to 30 hours. The stirring time of step b) may be up to 100, preferably up to 50 and more preferably up to 10 hours. The temperature in step b) may be at about room temperature, preferably 20 to 30° C. Removal of solvent and water is preferably carried out at about room temperature while applying vacuum, a dry inert gas flow or both. The same methods may be applied for drying the filtrate.

Preparation of Crystal Form C

In another aspect, disclosed herein is a process for the preparation of N-desmethylclozapine Form C comprising dissolving solid N-desmethylclozapine in a polar solvent or solvent mixture and slowly evaporating said solvent or solvent mixture at room temperature to dryness. A preferred solvent mixture is ethanol and methyl-isobutyl ketone (5:1 to 1:5 v/v). The concentration of N-desmethylclozapine in the solution may be from 3 to 30 and preferably 5 to 20 percent by weight. Evaporation may be carried out under reduced pressure and/or by exposure to a dry inert gas flow such as dry nitrogen. The flow rate of the inert gas may range from 1 to 20 and preferably 5 to 15 mL/minute.

Preparation of Crystal Form D

While N-desmethylclozapine Form B is very stable under normal conditions and at ambient temperatures, it is unstable at elevated temperatures or under dry nitrogen. Under these conditions, N-desmethylclozapine Form B loses its water and is transformed into N-desmethylclozapine Form D.

Thus, in another aspect, disclosed herein is a process for the preparation of N-desmethylclozapine Form D comprising heating N-desmethylclozapine Form B to temperatures of 35 to 80° C. The treating temperature is preferably from 40 to 70° C. The exposure time to heat may be from 1 to 5 and preferably 2 to 4 hours.

Preparation of Crystal Form E

In a further aspect, disclosed herein is a process for the preparation of N-desmethylclozapine Form E comprising dissolution of solid N-desmethylclozapine in an aliphatic ether and slowly evaporating said solvent or solvent mixture at room temperature to dryness. A preferred solvent is tetrahydrofuran. The concentration of N-desmethylclozapine in the solution may be from 3 to 25 and preferably 5 to 20 percent by weight. Evaporation may be carried out under reduced pressure and/or by exposure to a dry inert gas flow such as dry nitrogen. The flow rate of the inert gas may range from 1 to 20 and preferably 5 to 15 mL/minute.

Pharmaceutical Compositions

In another aspect, the present disclosure relates to a pharmaceutical composition comprising a physiologically acceptable carrier, diluent, or excipient, or a combination thereof; and N-desmethylclozapine in crystalline Form A, Form B, Form C, Form D, or Form E.

The term “pharmaceutical composition” refers to a mixture of a compound of the invention with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly in the renal or cardiac area, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions of the present invention may be manufactured in a manner that is itself known, e.g., by means of conventional mixing, dissolving, granulating, dragee-making, levigating, emulsifying, encapsulating, entrapping or tabletting processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the present invention will use those same dosages, or dosages that are between about 0.1% and 500%, more preferably between about 25% and 250% of the established human dosage. Where no human dosage is established, as will be the case for newly-discovered pharmaceutical compounds, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions of the present invention or a pharmaceutically acceptable salt thereof calculated as the free base, the composition being administered 1 to 4 times per day. Alternatively the compositions of the invention may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. Suitably the compounds will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

The amount of crystal forms of N-desmethylclozapine substantially depends on type of formulation and desired dosages during administration time periods. The amount in an oral formulation may be from 0.1 to 500 mg, preferably from 0.5 to 300 mg, and more preferably from 1 to 100 mg.

Oral formulations may be solid formulations such as capsules, tablets, pills and troches, or liquid formulations such as aqueous suspensions, elixirs and syrups. Solid and liquid formulations encompass also incorporation of crystal forms of N-desmethylclozapine according to the invention into liquid or solid food. Liquids also encompass solutions of N-desmethylclozapine for parenteral applications such as infusion or injection.

The crystal form according to the invention may be directly used as powder (micronized particles), granules, suspensions or solutions, or it may be combined together with other pharmaceutically acceptable ingredients in admixing the components and optionally finely divide them, and then filling capsules, composed for example from hard or soft gelatine, compressing tablets, pills or troches, or suspend or dissolve them in carriers for suspensions, elixirs and syrups. Coatings may be applied after compression to form pills.

Pharmaceutically acceptable ingredients are well known for the various types of formulation and may be for example binders such as natural or synthetic polymers, excipients, lubricants, surfactants, sweetening and flavouring agents, coating materials, preservatives, dyes, thickeners, adjuvants, antimicrobial agents, antioxidants and carriers for the various formulation types.

Examples for binders are gum tragacanth, acacia, starch, gelatine, and biological degradable polymers such as homo- or co-polyesters of dicarboxylic acids, alkylene glycols, polyalkylene glycols and/or aliphatic hydroxyl carboxylic acids; homo- or co-polyamides of dicarboxylic acids, alkylene diamines, and/or aliphatic amino carboxylic acids; corresponding polyester-polyamide-co-polymers, polyanhydrides, polyorthoesters, polyphosphazene and polycarbonates. The biological degradable polymers may be linear, branched or crosslinked. Specific examples are poly-glycolic acid, poly-lactic acid, and poly-d,l-lactide/glycolide. Other examples for polymers are water-soluble polymers such as polyoxaalkylenes (polyoxaethylene, polyoxapropylene and mixed polymers thereof, polyacrylamides and hydroxylalkylated polyacrylamides, poly-maleic acid and esters or -amides thereof, poly-acrylic acid and esters or -amides thereof, poly-vinylalcohol und esters or -ethers thereof, poly-vinylimidazole, poly-vinylpyrrolidon, and natural polymers like chitosan.

Examples for excipients are phosphates such as dicalcium phosphate.

Examples for lubricants are natural or synthetic oils, fats, waxes, or fatty acid salts like magnesium stearate.

Surfactants may be anionic, anionic, amphoteric or neutral. Examples for surfactants are lecithin, phospholipids, octyl sulfate, decyl sulfate, dodecyl sulfate, tetradecyl sulfate, hexadecyl sulfate and octadecyl sulfate, Na oleate or Na caprate, 1-acylaminoethane-2-sulfonic acids, such as 1-octanoylaminoethane-2-sulfonic acid, 1-decanoylaminoethane-2-sulfonic acid, 1-dodecanoylaminoethane-2-sulfonic acid, 1-tetradecanoylaminoethane-2-sulfonic acid, 1-hexadecanoylaminoethane-2-sulfonic acid, and 1-octadecanoylaminoethane-2-sulfonic acid, and taurocholic acid and taurodeoxycholic acid, bile acids and their salts, such as cholic acid, deoxycholic acid and sodium glycocholates, sodium caprate or sodium laurate, sodium oleate, sodium lauryl sulphate, sodium cetyl sulphate, sulfated castor oil and sodium dioctylsulfosuccinate, cocamidopropylbetaine and laurylbetaine, fatty alcohols, cholesterols, glycerol mono- or -distearate, glycerol mono- or -dioleate and glycerol mono- or -dipalmitate, and polyoxyethylene stearate.

Examples for sweetening agents are sucrose, fructose, lactose or aspartam.

Examples for flavouring agents are peppermint, oil of wintergreen or fruit flavours like cherry or orange flavour.

Examples for coating materials are gelatine, wax, shellac, sugar or biological degradable polymers.

Examples for preservatives are methyl or propylparabens, sorbic acid, chlorobutanol, phenol and thimerosal.

Examples for adjuvants are fragrances.

Examples for thickeners are synthetic polymers, fatty acids and fatty acid salts and esters and fatty alcohols.

Examples for antioxidants are vitamins, such as vitamin A, vitamin C, vitamin D or vitamin E, vegetable extracts or fish oils.

Examples for liquid carriers are water, alcohols such as ethanol, glycerol, propylene glycol, liquid polyethylene glycols, triacetin and oils. Examples for solid carriers are talc, clay, microcrystalline cellulose, silica, alumina and the like.

The formulation according to the invention may also contain isotonic agents, such as sugars, buffers or sodium chloride.

The hydrate Form B may also be formulated as effervescent tablet or powder, which disintegrate in an aqueous environment to provide a drinking solution.

A syrup or elixir may contain the polymorph of the invention, sucrose or fructose as sweetening agent a preservative like methylparaben, a dye and a flavouring agent.

Slow release formulations may also be prepared from the polymorph disclosed herein in order to achieve a controlled release of the active agent in contact with the body fluids in the gastro intestinal tract, and to provide a substantial constant and effective level of the active agent in the blood plasma. The crystal form may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

The crystal forms of this invention are also useful for administering a combination of therapeutic effective agents to an animal. Such a combination therapy can be carried out in using at least one further therapeutic agent which can be additionally dispersed or dissolved in a formulation.

The crystal form of this invention and its formulations respectively can be also administered in combination with other therapeutic agents that are effective to treat a given condition to provide a combination therapy.

The crystal form and the pharmaceutical composition according to the invention are highly suitable for effective treatment of neuropsychiatric diseases including psychosis, affective disorders, dementia, neuropathic pain and glaucoma.

Disclosed herein is a method of delivering N-desmethylclozapine in crystalline Form A, Form B, Form C, Form D, or Form E to a host, comprising administering to a host an effective amount of a N-desmethylclozapine in crystalline Form A, Form B, Form C, Form D, or Form E.

Further disclosed herein is the use of N-desmethylclozapine in crystalline Form A, Form B, Form C, Form D, or Form E for the manufacture of a medicament useful in the treatment of neuropsychiatric diseases including psychosis, affective disorders, dementia, neuropathic pain and glaucoma.

Disclosed herein is a method of treating psychosis comprising: identifying a subject suffering from one or more symptoms of psychosis; and contacting the subject with a therapeutically effective amount of N-desmethylclozapine in crystalline Form A, Form B, Form C, Form D, or Form E; whereby the one or more symptoms of psychosis are ameliorated. In one embodiment, the subject is human. In some embodiments, the therapeutically effective amount of N-desmethylclozapine is administered as a single dose. In other embodiments, the therapeutically effective amount of N-desmethylclozapine is administered as a plurality of doses. In one embodiment, the method further comprises contacting the subject with an additional therapeutic agent. In one embodiment, the subject is contacted with the additional therapeutic agent subsequent to the contacting with N-desmethylclozapine. In another embodiment, the subject is contacted with the additional therapeutic agent prior to the contacting with N-desmethylclozapine. In still another embodiment, the subject is contacted with the additional therapeutic agent substantially simultaneously with N-desmethylclozapine. In some embodiments, the additional therapeutic agent is selected from the group consisting of monoamine repuptake inhibitiors, selective serotonin reuptake inhibitors, norepinephrine reuptake inhibitors, dual serotonin and norepinephrine reupake inhibitors, dopamine agonists, antipsychotic agents, inverse serotonin agonists, serotonin antagonists, serotonin 2 inverse agonists, serotonin 2 antagonists, serotonin1A agonists, antiepileptic and peripherally acting muscarinic antagonists.

Also disclosed herein is a method of treating affective disorders comprising: identifying a subject suffering from one or more symptoms of an affective disorder; and administering a therapeutically effective amount of N-desmethylclozapine in crystalline Form A, Form B, Form C, Form D, or Form E to the subject, whereby the one or more symptoms of the affective disorder are ameliorated. In one embodiment, the subject is human. In one embodiment, the affective disorder is depression. In another embodiment, the affective disorder is mania. In some embodiments, the therapeutically effective amount of N-desmethylclozapine is administered as a single dose. In other embodiments, the therapeutically effective amount of N-desmethylclozapine is administered as a plurality of doses. In one embodiment, the method further comprises administering to the subject an additional therapeutic agent. In one embodiment, the subject is contacted with the additional therapeutic agent subsequent to the contacting with N-desmethylclozapine. In another embodiment, the subject is contacted with the additional therapeutic agent prior to the contacting with N-desmethylclozapine. In still another embodiment, the subject is contacted with the additional therapeutic agent substantially simultaneously with N-desmethylclozapine. In some embodiments, the additional therapeutic agent is selected from the group consisting of monoamine reuptake inhibitors, selective serotonin reuptake inhibitors, norepinephrine reuptake inhibitors, dual serotonin and norepinephrine reuptake inhibitors, dopamine agonists, antipsychotic agents, inverse serotonin agonists, serotonin antagonists, serotonin 2 inverse agonists, serotonin 2 antagonists, serotonin1A agonists, antiepileptic and peripherally acting muscarinic antagonists.

Also disclosed herein is a method of treating dementia, comprising: identifying a subject suffering from one or more symptoms of dementia; and administering a therapeutically effective amount of N-desmethylclozapine in crystalline Form A, Form B, Form C, Form D, or Form E to said subject, whereby a desired clinical effect is produced. In one embodiment, the subject is human. In some embodiments, the therapeutically effective amount of N-desmethylclozapine is administered as a single dose. In other embodiments, the therapeutically effective amount of N-desmethylclozapine is administered as a plurality of doses. In one embodiment, the dementia manifests as a cognitive impairment. In another embodiment, the dementia manifests as a behavioral disturbance. In one embodiment, the method further comprises administering to the subject an additional therapeutic agent. In one embodiment, the subject is contacted with the additional therapeutic agent subsequent to the contacting with N-desmethylclozapine. In another embodiment, the subject is contacted with the additional therapeutic agent prior to the contacting with N-desmethylclozapine. In still another embodiment, the subject is contacted with the additional therapeutic agent substantially simultaneously with N-desmethylclozapine. In some embodiments, the additional therapeutic agent is selected from the group consisting of monoamine reuptake inhibitors, selective serotonin reuptake inhibitors, norepinephrine reuptake inhibitors, dual serotonin and norepinephrine reuptake inhibitors, dopamine agonists, antipsychotic agents, inverse serotonin agonists, serotonin antagonists, serotonin 2 inverse agonists, serotonin 2 antagonists, serotonin1A agonists, antiepileptic and peripherally acting muscarinic antagonists.

Also disclosed herein is a method of treating neuropathic pain comprising: identifying a subject suffering from one or more symptoms of neuropathic pain; and contacting said subject with a therapeutically effective amount of N-desmethylclozapine in crystalline Form A, Form B, Form C, Form D, or Form E, whereby the symptoms of neuropathic pain are reduced. In one embodiment, the subject is human. In some embodiments, the therapeutically effective amount of N-desmethylclozapine is administered as a single dose. In other embodiments, the therapeutically effective amount of N-desmethylclozapine is administered as a plurality of doses. In one embodiment, the method further comprises contacting the subject with an additional therapeutic agent. In one embodiment, the subject is contacted with the additional therapeutic agent subsequent to the contacting with N-desmethylclozapine. In another embodiment, the subject is contacted with the additional therapeutic agent prior to the contacting with N-desmethylclozapine. In still another embodiment, the subject is contacted with the additional therapeutic agent substantially simultaneously with N-desmethylclozapine. In some embodiments, the additional therapeutic agent is selected from the group consisting monoamine reuptake inhibitors, selective serotonin reuptake inhibitors, norepinephrine reuptake inhibitors, dual serotonin and norepinephrine reuptake inhibitors, dopamine agonists, antipsychotic agents, inverse serotonin agonists, serotonin antagonists, serotonin 2 inverse agonists, serotonin 2 antagonists, serotonin1A agonists, antiepileptic and peripherally acting muscarinic antagonists.

Also disclosed herein is a method of treating glaucoma comprising: identifying a subject suffering from one or more symptoms of glaucoma; and contacting said subject with a therapeutically effective amount of N-desmethylclozapine in crystalline Form A, Form B, Form C, Form D, or Form E, whereby the symptoms of glaucoma are reduced. In one embodiment, the subject is human. In some embodiments, the therapeutically effective amount of N-desmethylclozapine is administered as a single dose. In other embodiments, the therapeutically effective amount of N-desmethylclozapine is administered as a plurality of doses. In some embodiments, the symptoms of glaucoma are selected from the group consisting of elevated intraocular pressure, optic nerve damage, and decreased field of vision. In one embodiment, the method further comprises contacting the subject with an additional therapeutic agent. In one embodiment, the subject is contacted with the additional therapeutic agent subsequent to the contacting with N-desmethylclozapine. In another embodiment, the subject is contacted with the additional therapeutic agent prior to the contacting with N-desmethylclozapine. In still another embodiment, the subject is contacted with the additional therapeutic agent substantially simultaneously with N-desmethylclozapine. In some embodiments, the additional therapeutic agent is selected from the group consisting of monoamine reuptake inhibitors, selective serotonin reuptake inhibitors, norepinephrine reuptake inhibitors, dual serotonin and norepinephrine reuptake inhibitors, dopamine agonists, antipsychotic agents, inverse serotonin agonists, serotonin antagonists, serotonin 2 inverse agonists, serotonin 2 antagonists, serotonin1A agonists, antiepileptics, prostenoids and alpha and beta adrenergic agonists.

Also disclosed herein is a pharmaceutical composition comprising a pharmaceutically effective amount of N-desmethylclozapine in crystalline Form A, Form B, Form C, Form D, or Form E and an additional therapeutic agent. In some embodiments, the additional therapeutic agent is selected from the group consisting of monoamine reuptake inhibitors, selective serotonin reuptake inhibitors, norepinephrine reuptake inhibitors, dual serotonin and norepinephrine reuptake inhibitors, dopamine agonists, antipsychotic agents, inverse serotonin agonists, serotonin antagonists, serotonin 2 inverse agonists, serotonin 2 antagonists, serotonin1A agonists, antiepileptic and peripherally acting muscarinic antagonists. In some embodiments, the additional therapeutic agent is selected from the group consisting of a phenothiazine, phenylbutylpiperadine, debenzapine, benzisoxidil, and salt of lithium. In some embodiments, the additional therapeutic gent is selected from the group consisting of chlorpromazine (Thorazine®), mesoridazine (Serentil®), prochlorperazine (Compazine®), thioridazine (Mellaril®), haloperidol (Haldol®), pimozide (Orap®), clozapine (Clozaril®), loxapine (Loxitane®), olanzapine (Zyprexa®), quetiapine (Seroquel®), risperidone (Risperidal®), ziprasidone (Geodon®), lithium carbonate, Aripiprazole (Abilify), Clozapine, Clozaril, Compazine, Etrafon, Geodon, Haldol, Inapsine, Loxitane, Mellaril, Moban, Navane, Olanzapine (Zyprexa), Orap, Permitil, Prolixin, Phenergan, Quetiapine (Seroquel), Reglan, Risperdal, Serentil, Seroquel, Stelazine, Taractan, Thorazine, Triavil, Trilafon, Zyprexa, and pharmaceutically acceptable salts thereof. In some embodiments the selective serotonin reuptake inhibitor is selected from the group consisting of fluoxetine, fluvoxamine, sertraline, paroxetine, citalopram, escitalopram, sibutramine, duloxetine, venlafaxine, and pharmaceutically acceptable salts and prodrugs thereof. In some embodiments, the norepinephrine reuptake inhibitor is selected from the group consisting of thionisoxetine and reboxetine. In some embodiments, the dual serotonin and norepinephrine reuptake inhibitor is selected from the group consisting of duloxetine, milnacripran and fluvoxamine. In some embodiments, the dopamine agonist is selected from the group consisting of cabergoline, amantadine, lisuride, pergolide, ropinirole, pramipexole, L-DOPA and bromocriptine. In one embodiment, the inverse serotonin agonists selected from the group consisting of N-(1-methylpiperidin-4-yl)-N-(4-flourophenylmethyl)-N′-(4-(2-methylpropyloxy)phenylmethyl)carbamide, MDL 100,907, SR-43694B (eplivanserin), ritanserin, ketanserin, mianserin, cinanserin, mirtazepine, cyproheptadine and cinnarizine.

One embodiment of the present invention includes, a method of treating cognitive impairment comprising identifying a subject in need of improvement of cognition and administering an amount of N-desmethylclozapine in crystalline Form A, Form B, Form C, Form D, or Form E to said subject, which is therapeutically effective in improving the cognition of said subject.

In some aspects of this embodiment, the subject is human. In some aspects of this embodiment, the therapeutically effective amount of N-desmethylclozapine in crystalline Form A, Form B, Form C, Form D, or Form E is administered as a single dose. In other aspects of this embodiment, the therapeutically effective amount of N-desmethylclozapine is administered as a plurality of doses.

In further aspects of this embodiment, the method further comprises contacting the subject with an additional therapeutic agent. For example, the subject may be contacted with said additional therapeutic agent subsequent to said contacting with N-desmethylclozapine in crystalline Form A, Form B, Form C, Form D, or Form E. Alternatively, the subject may be contacted with said additional therapeutic agent prior to said contacting with N-desmethylclozapine.

In some cases, the subject is contacted with said additional therapeutic agent substantially simultaneously with N-desmethylclozapine. In some cases, the additional therapeutic agent is selected from the group consisting of monoamine reuptake inhibitors, selective serotonin reuptake inhibitors, norepinephrine reuptake inhibitors, dual serotonin and norepinephrine reuptake inhibitors, dopamine agonists, antipsychotic agents, inverse serotonin agonists, serotonin antagonists, serotonin 2 inverse agonists, serotonin 2 antagonists, serotonin1A agonists, antiepileptic and peripherally acting muscarinic antagonists. In some aspects of this embodiment, the subject suffers from a condition selected from the group consisting of hallucinations, delusions, disordered thought, behavioral disturbance, aggression, suicidality, mania, anhedonia, flattening of affect, affective disorders, depression, mania, dementia, neuropathic pain, glaucoma and two or more any of the foregoing conditions.

Another embodiment of the present invention includes method of ameliorating at least one symptom of a condition where it is beneficial to increase the level of activity of an M1 muscarinic receptor comprising determining that a subject would benefit from an increased level of activity of an M1 muscarinic receptor and administering an amount of N-desmethylclozapine in crystalline Form A, Form B, Form C, Form D, or Form E which is therapeutically effective to increase the level of activity of the M1 muscarinic receptor and to ameliorate said at least one symptom to the subject. In some aspects of this embodiment, the therapeutically effective amount of N-desmethylclozapine is administered as a single dose. In other aspects of this embodiment, the therapeutically effective amount of N-desmethylclozapine is administered as a plurality of doses. In further aspects of this embodiment, the method further comprises contacting the subject with an additional therapeutic agent. For example, the subject may be contacted with said additional therapeutic agent subsequent to said contacting with N-desmethylclozapine. Alternatively, the subject may be contacted with said additional therapeutic agent prior to said contacting with N-desmethylclozapine. In some cases, the subject is contacted with said additional therapeutic agent substantially simultaneously with N-desmethylclozapine. In some cases, the additional therapeutic agent is selected from the group consisting of monoamine reuptake inhibitors, selective serotonin reuptake inhibitors, norepinephrine reuptake inhibitors, dual serotonin and norepinephrine reuptake inhibitors, dopamine agonists, antipsychotic agents, inverse serotonin agonists, serotonin antagonists, serotonin 2 inverse agonists, serotonin 2 antagonists, serotonin1A agonists, antiepileptic and peripherally acting muscarinic antagonists. In some aspects of this embodiment, the subject suffers from a condition selected from the group consisting of hallucinations, delusions, disordered thought, behavioral disturbance, aggression, suicidality, mania, anhedonia, flattening of affect, affective disorders, depression, mania, dementia, neuropathic pain, glaucoma and two or more any of the foregoing conditions.

### EXAMPLES

A) Preparation of N-Desmethylclozapine

### Example A1

Coupling of Piperazine

A 100 L enamelled reactor is charged with anisole (16 L) at 20° C. inner temperature and TiCl₄ (1.064 kg, 1.37 equivalents) is added. The feed tank is rinsed with anisole (210 mL). Piperazine (2.113 kg, 6 equivalents) are added and the resulting brown suspension is warmed to 55° C. inner temperature. No significant exothermic reaction is observed. The compound of formula II (1.001 kg, 1 equivalent) is added at 55-60° C. inner temperature in portions over 30 minutes. An exothermic reaction occurs after the addition of the first portion, and the inner temperature raises to 65° C. (external cooling at −5° C. is applied). After the addition is complete, the brown reaction mixture is heated to 125° C. jacket temperature (120-124° C. inner temperature) and stirred for 4.5 h at this temperature. In process control by HPLC shows a conversion of 99%.

Filtration of Titanium Salts

The reaction mixture is cooled to −2° C. inner temperature. NaOH (30%, 2.4 L, 5.5 equivalents) is added at this temperature over 80 minutes (an exothermic reaction occurs). After the addition is complete, the resulting suspension is warmed to 22° C. inner temperature over 60 minutes. The titanium salts form a well filterable, granulated solid which is filtered off over a pad of celite (10 L pressure filter). The reactor and the filter cake are washed with t-butyl methyl ether (TBME, 10 L). The brown filtrate (29 L) is washed with NaOH (0.1 M, 7 L).

Extractive Workup

The organic phase is extracted with HCl in three portions (1 M, 8+7+3.5 L). The acidic aqueous layers are combined and washed with TBME (4.5 L). TBME (6.5 L) is added to the aqueous phase and the pH is adjusted to 13 by the addition of NaOH (30%, 2.5 L). The organic layer is separated and the aqueous layer is extracted with TBME (6 L). The combined TBME-layers are washed with half-saturated brine in two portions (2×4 L), then filtered over a 10 L pressure filter charged with Na₂SO4 (3.97 kg). The filter cake is washed with TBME in portions (9 L in total).

Crystallization from TBME

The combined filtrates (approximately 25 L) are concentrated under reduced pressure (350 mbar, 45° C. jacket temperature) to a residual volume of approximately 1.5 L. The residual brown thick solution is warmed to 40° C. inner temperature, then cooled to −1° C. A thick yellow suspension is formed, which is diluted with TBME (2 L). Stirring at this temperature is continued for approximately 60 minutes. The suspension is filtered off (10 L pressure filter, 1200 mbar). The solids are dried on a rotary evaporator under reduced pressure at 80° C. for approximately 7 h. The operation yields 542.11 g of a yellow solid, containing approximately 3.75 percent by weight of TBME as determined by NMR.

Water Slurry and Final Drying

The yellow solid is suspended in water (5.5 L) and the mixture is stirred 20 hours at 22° C. inner temperature. The solid is filtered off (10 L pressure filter, 1200 mbar). The filter cake is rinsed with water in portions (in total 4 L). The product is dried for 3 days on the filter in a stream of nitrogen, and then further dried under reduced pressure (<20 mbar), at 60° C. bath temperature for 5 hours to yield 427.71 g of N-desmethylclozapine as a yellow solid (33% based on the amount of 8-chloro-11-oxo-10,11-dihydro-5H-dibenzo-1,4-diazepin). The melting range of the product is 110.6-124.1° C. and the solid product is a non-crystalline and amorphous product, as shown by powder X-ray diffraction measurement.

### Example A2

A 640 L enamelled reactor is charged with anisole (390 L) at 20° C. inner temperature and TiCl₄ (20.4 kg, 12 L, 1.1 equivalents) is added. The feed tank is rinsed with anisole (5 L) to remove all TiCl₄. Piperazine (50.66 kg, 6 equivalents) is added and the resulting brown suspension is warmed to 55° C. inner temperature. At 54° C. inner temperature an exothermic reaction is observed and the inner temperature raises to 65° C. External cooling at 20° C. is applied. 8-chloro-11-oxo-10,11-dihydro-5H-dibenzo-1,4-diazepin (compound of formula II, 23.9 kg, 1 equivalent) is added at 55-60° C. inner temperature in portions over 40 minutes. After the addition is complete, the brown reaction mixture is heated to 125° C. jacket temperature (120-124° C. inner temperature) and stirred for 4.5 h at this temperature (thick brown suspension). In process control by HPLC shows a conversion of 99%.

Filtration of Titanium Salts

The reaction mixture is cooled to −2° C. inner temperature. NaOH (30%, 47 L, 4.8 equivalents) is added over 5.5 hours, keeping the inner temperature below 5° C. by external cooling at −30° C. The reaction mixture is stirred at 1° C. for approximately 8 hours, then warmed to 20° C. over approximately 3 hours (thick green suspension). The solid is filtered off over a pad of celite (using two 50 L pressure filters). A total of approximately 500 L of TBME is used for the filtration and wash. The 840 L combined filtrates are washed in two portions with 75 L of 0.1 M NaOH each.

Extractive Workup

The organic phase is divided into two parts of approximately 420 L each. Each part is extracted with HCl (1 M, 2×73 L+24 L) in three portions. All acidic aqueous layers are combined. TBME (107 L) is added and the mixture is stirred for approximately 20 minutes at 20° C. A precipitate forms. Addition of water (210 L) and TBME (50 L) did not improve layer separation. NaOH (30%, 50 L) is added to adjust the pH to 14. The solid dissolved and the layers are separated. The product-containing brown TBME-layer is separated and the aqueous layer is extracted with TBME (147 L). The organic layers are combined and washed with half-saturated brine (2×73 L) in two portions. A precipitate forms during the washing with the second portion, and layer separation is not possible. Additional TBME (145 L) is added under stirring, but the solid does not dissolve. Ethyl acetate (225 L) is added to the mixture in the reactor, but the solid does not dissolve completely. The mixture is divided into two parts: one part consisting of a three-phase mixture of water, organic phase and precipitate, and a second part consisting of a clear organic phase. The reactor is charged again with the first part, and ethyl acetate (135 L) and water (40 L) were added. The solids do not dissolve. TBME (74 L) and NaOH (1.5 M, 105 L) are added to the mixture in the reactor and stirring is continued. The solids still do not dissolve. The mixture is filtered off (170 L pressure filter) to obtain a yellow filter cake (8.4 kg of wet material) and a two-phase filtrate, which can be separated very well. The organic phase is combined with the previously obtained clear organic phase (in total 828 L) and concentrated under reduced pressure (330-230 mbar) at 40-45° C. jacket temperature. The product precipitates in the mixture during the distillation. When a residual volume of 500 L is reached, the previously filtered solid (8.4 kg) is added to the mixture in the reactor. A total of 700 L of solvents are distilled off.

Crystallization from Ethyl Acetate/TBME

The thick yellow suspension is diluted with ethyl acetate (32 L) and TBME (60 L). The suspension is heated to reflux, then cooled to 5° C. inner temperature over 3 hours and stirred at this temperature for further 45 minutes. The solid is filtered off (170 L pressure filter, 1-3 bar pressure). The wet filter cake is dried under a stream of nitrogen over 96 hours. This yields 23.58 kg of yellow solid.

Crystallization of Impurities from Ethyl Acetate/TBME

In an effort to selectively crystallize out impurities, a 640L-reactor is charged with the above solid (23.58 kg) and a mixture of TBME/ethyl acetate (10:1, 472 L) is added. The resulting suspension is heated to reflux (jacket temperature: 70° C.) and stirred at this temperature for 1 hour. The suspension is cooled to 0° C. over 3 h. The yellow solid (18.57 kg) is filtered off. The product crystallizes with impurities.

Acetic Acid Extraction

The 160 L enamelled reactor is charged with a fraction of the above solid (2.500 kg out of the 18.57 kg) and with dichloromethane (50 L). The resulting suspension is stirred at room temperature for 50 minutes. Aqueous acetic acid (5% v/v, 22 L) is added and stirring is continued for 15 minutes. The aqueous layer is separated and the organic layer is extracted a second time with aqueous acetic acid (5% v/v, 10 L). Dichloromethane (25 L) is added to the combined acidic aqueous layers and the pH is adjusted to 14 by the addition of NaOH (30%, 4 L). The brown organic layer is separated and the aqueous layer is extracted with dichloromethane (13 L). The combined organic layers are washed with water (13 L). The organic phase is dried over Na₂SO₄ (16.9 kg), filtered through an inline filter and washed with dichloromethane (15 L).

Crystallization from Dichloromethane/Methylcyclohexane

The filtrates are concentrated to a residual volume of 10 L. The resulting brown solution is heated to reflux and methylcyclohexane (MCH, 15 L) is added under reflux. The resulting clear yellow solution is cooled slowly to −7° C. jacket temperature over 7 hours to obtain a yellow suspension, which is stirred at −5° C. inner temperature for further 60 minutes. The solid is filtered off, washed with cooled MCH (10 L) and dried in a stream of nitrogen for 2 h. Drying is continued on a rotary evaporator under reduced pressure at 80° C. for 3 hours.

Water Slurry and Final Drying

A 1601 reactor is charged with the above crystallized product (1.5 kg) and water (16 L), and the yellow suspension is stirred at 25° C. for 1.5 hours. The suspension is filtered off over 20 hours. The filter cake is washed with water (10 L+5 L) and dried on the filter under a stream of nitrogen for 24 hours. Drying is continued on a rotary evaporator at 80° C. bath temperature (<2 mbar) for 17 hours to give 1.363 kg of the product as a yellow solid (4.5% yield based on 23.9 kg 8-chloro-11-oxo-10,11-dihydro-5H-dibenzo-1,4-diazepin). The melting range of the product is 176.4-177.6° C. and the solid product is crystalline and a mixture of crystal forms A and B (monohydrate) as shown by powder X-ray diffraction and comparison of the pattern with those of pure crystal forms A and B. The solid product is hereinafter called “product A2”.

B) Preparation of Crystal Form A

### Example B1

100 mg of product A2 are suspended in 1.5 mL ethyl acetate and heated to 60° C. A clear, yellow solution forms, which is cooled down to 5° C. and stored at this temperature for 3 days. Since no crystallization is observed upon storage in a refrigerator, 1.5 mL of heptane are added at room temperature and a solid yellow product precipitates. The solid is filtered off and dried at room temperature in a dry air flow for 1 day. The dried crystalline solid is crystal form A.

### Example B2

A suspension of 80 mg product A2 in 1.5 mL acetonitrile is heated to 60° C. A clear, yellow solution forms, which is cooled down to 5° C. and stored at this temperature for 3 days. The formed crystalline precipitate is filtered off and dried at room temperature in a dry air flow for 1 day. The dried crystalline solid is crystal form A.

### Example B3

250 mg of product A2 are suspended in 4.0 mL of heptane/ethyl acetate (3:1) and heated to 60° C. A yellow solution forms, which is filtered and then cooled down to 20° C. The precipitate is stirred at 20° C. for about 2 hours, filtered off and dried at room temperature in a dry air flow for 1 day. The dried crystalline solid is crystal form A. Yield: 139 mg form A The X-ray powder diffraction pattern is shown in FIG. 1 and the characteristic peaks in 2 theta with the corresponding d-spacing values in Å are given in table 1. The melting point is determined by DSC to be 177° C., and the enthalpy of fusion is about 96 J/g.

### Example B4

300 mg of product A2 are suspended in 10.0 mL of acetonitrile and heated to 60° C. A yellow solution forms, which is filtered. The volume is reduced to about 4.0 mL in an evaporator at 45° C. The obtained dark yellow suspension is cooled down to room temperature and stirred for about two days. The precipitate is filtered off and then dried at 40° C. in a dry air flow for 4 hours. The dried crystalline solid is crystal form A.

### Example B5

154 mg of product A2 are suspended in 3.0 mL of heptane and heated to 60° C. 1.0 mL ethanol is then added to obtain a clear solution. The solution is cooled to room temperature, but no crystallization occurs. 3.0 mL of heptane are added and half of the volume is evaporated under a dry nitrogen stream, whereby a crystalline precipitate is formed at room temperature. The crystalline solid is filtered of after 1 day storage and dried at 40° C. in a dry air flow for 4 hours. The dried crystalline solid is crystal form A.

C) Preparation of Crystal Form B (Monohydrate)

### Example C1

154 mg of product A2 are dissolved in 5.0 mL acetonitrile at room temperature and 12 mL water are added. The formed suspension is stored at 5° C. for 3 days, but no crystallization occurs. The solvent and water is evaporated under nitrogen and the residue is dried in a dry air flow at room temperature for 8 hours. The obtained product shows in a thermogravimetric experiment a water loss of 5.3 percent by weight, indicating formation of a crystalline monohydrate of N-desmethylclozapin. The X-ray powder diffraction pattern is shown in FIG. 2 and the characteristic peaks in 2 theta with the corresponding d-spacing values in Å are given in table 2. The melting point is determined by DSC to be 149° C. with an enthalpy of fusion of about 135 J/g.

### Example C2

60 mg of product A2 are suspended in 2 mL water and stirred at 23° C. for 22 hours. The solid is filtered off and dried in a dry air flow at room temperature for 8 hours. The dried crystalline solid is crystal form B.

### Example C3

100 mg of product A2 are suspended in 1.5 mL of water/methanol (9:1 v/v) and stirred at 23° C. for 22 hours. The solid is filtered off and dried inair at room temperature for 8 hours. The obtained crystalline solid is crystal form B.

D) Preparation of Crystal Form C

### Example D1

450 mg of product A2 are dissolved in a mixture of 3.0 mL ethanol and 2.0 mL methyl-isobutyl ketone. The obtained solution is filtered and the solvent mixture is slowly evaporated under dry nitrogen at a flow of about 10 mL/min at room temperature. The obtained solid is investigated by powder X-ray diffraction and shows that a crystal form C is obtained. The obtained form C apparently contains some amorphous material. The X-ray powder diffraction pattern is shown in FIG. 4 and the characteristic peaks in 2 theta with the corresponding d-spacing values in Å are given in table 4.

E) Preparation of Crystal Form D

### Example E1

About 40 mg of crystal form B prepared according to example C3 are filled into a powder X-ray diffraction sample holder and are treated at room temperature under a slight flow of dry nitrogen (about 30 mL/min) in a closed container for 6 days. A new crystal form D is obtained. The X-ray powder diffraction pattern recorded under dry nitrogen is shown in FIG. 4 and the characteristic peaks in 2 theta with the corresponding d-spacing values in Å are given in table 4.

### Example E2

126 mg of crystal form B prepared according to example C3 are treated for 3 hours under reduced pressure (1 mbar) at 45° C. The dried crystalline solid is investigated by Raman spectroscopy immediately after preparation and is identified as crystal form D.

### Example E3

1009 mg of crystal form B prepared according to example C3 are dried under vacuum at 80° C. for about 3 hours. The dried crystalline solid is investigated by Raman spectroscopy immediately after preparation and is identified as a mixture containing about 90% of crystal form D and about 10% of crystal form A.

F) Preparation of Crystal Form E

### Example F1

148 mg of product A2 are dissolved in 2.0 mL of tetrahydrofurane (THF) and the obtained solution is filtered. The solvent is then slowly evaporated under dry nitrogen at a flow of about 10 mL/min at room temperature. The obtained solid is investigated by powder X-ray diffraction and shows that a form E is obtained. The obtained form E apparently contains some amorphous material. The X-ray powder diffraction pattern is shown in FIG. 5 and the characterristic peaks in 2 theta with the corresponding d-spacing values in Å are given in table 5.

Experimental:

Powder X-ray Diffraction (PXRD): PXRD is performed on a Philips 1710 powder X-ray diffractometer using CuK_{α} radiation. D-spacings are calculated from the 2° values using the wavelength of 1.54060 Å. Generally, 2θ values are within an error of ±0.1-0.2°. The experimental error on the d-spacing values is therefore dependent on the peak location.

Forms B and D are characterized in a Philips X'Pert powder X-ray diffractometer using TTK sample holders obtained from Anton Paar, Inc. (Austria). PXRD patterns are collected in a closed measurement chamber under controlled relative humidity, or under dry nitrogen, respectively.

Differential Scanning Calorimetry: Perkin Elmer DSC 7 in gold sample pan sealed under nitrogen for characterization of form A and sealed under about 50% relative humidity for characterization of form B. Heating rate 10 K/min.

FT-Raman Spectroscopy: Bruker RFS100. Nd:YAG 1064 nm excitation, 100 mW laser power, Ge-detector, 64 scans, range 25-3500 cm⁻¹, 2 cm⁻¹ resolution.

What is claimed is:

1. A method of treating a disease or disorder comprising:
administering a therapeutically effective amount of at least one polymorph of crystalline N-desmethylclozapine to a subject suffering from said disease or disorder.

2. The method of claim 1, wherein the crystalline N-desmethylclozapine is Form A.

3. The method of claim 1, wherein the crystalline N-desmethylclozapine is Form B.

4. The method of claim 1, wherein the crystalline N-desmethylclozapine is Form C.

5. The method of claim 1, wherein the crystalline N-desmethylclozapine is Form D.

6. The method of claim 1, wherein the crystalline N-desmethylclozapine is Form E.

7. The method of claim 1, wherein the crystalline N-desmethylclozapine is Form F.

8. The method of claim 1, wherein the crystalline N-desmethylclozapine is a mixture of Form A and B.

9. The method of claim 1, wherein the subject is human.

10. The method of claim 1, wherein the disease or disorder is selected from the group consisting of pyschosis, an affective disorder, dementia, neuropathic pain, and glaucoma.

11. The method of claim 10, wherein the affective disorder selected from the group consisting of mania and depression.

12. The method of claim 10, wherein the dementia manifests as a cognitive impairment or a behavioral disturbance.

13. The method of claim 10, wherein the crystalline N-desmethylclozapine reduces one of symptoms of glaucoma selected from the group consisting of elevated intraocular pressure, optic nerve damage, and decreased field of vision.

14. The method of claim 1, further comprising an additional therapeutic agent.

15. The method of claim 14, wherein the additional therapeutic agent is selected from the group consisting of monoamine repuptake inhibitiors, selective serotonin reuptake inhibitors, norepinephrine reuptake inhibitors, dual serotonin and norepinephrine reupake inhibitors, dopamine agonists, antipsychotic agents, inverse serotonin agonists, serotonin antagonists, serotonin 2 inverse agonists, serotonin 2 antagonists, serotonin1A agonists, antiepileptic and peripherally acting muscarinic antagonists.

16. The method of claim 14, wherein the additional therapeutic agent is selected from the group consisting of phenothiazine, phenylbutylpiperadine, debenzapine, benzisoxidil, and salt of lithium.

17. The method of claim 14, wherein the additional therapeutic agent is selected from the group consisting of chlorpromazine (Thorazine®), mesoridazine (Serentil®), prochlorperazine (Compazine®), thioridazine (Mellaril®), haloperidol (Haldol®), pimozide (Orap®), clozapine (Clozaril®), loxapine (Loxitane®), olanzapine (Zyprexa®), quetiapine (Seroquel®), risperidone (Risperidal®), ziprasidone (Geodon®), lithium carbonate, Aripiprazole (Abilify), Clozapine, Clozaril, Compazine, Etrafon, Geodon, Haldol, Inapsine, Loxitane, Mellaril, Moban, Navane, Olanzapine (Zyprexa), Orap, Permitil, Prolixin, Phenergan, Quetiapine (Seroquel), Reglan, Risperdal, Serentil, Seroquel, Stelazine, Taractan, Thorazine, Triavil, Trilafon, Zyprexa, and pharmaceutically acceptable salts thereof.

18. A method of ameliorating at least one symptom of a condition where it is beneficial to increase the level of activity of an M1 muscarinic receptor comprising determining that a subject would benefit from an increased level of activity of an M1 muscarinic receptor and administering an amount of N-desmethylclozapine in crystalline Form A, Form B, Form C, Form D, Form E or mixtures thereof which is therapeutically effective to increase the level of activity of the M1 muscarinic receptor and to ameliorate at least one symptom to the subject.

19. The method of claim 18, wherein the symptom results from a condition selected from the group consisting of hallucinations, delusions, disordered thought, behavioral disturbance, aggression, suicidality, mania, anhedonia, flattening of affect, affective disorders, depression, mania, dementia, neuropathic pain, glaucoma and any combination thereof.
